(19) United States
(12) Patent Application Publication
(10) Pub. No.: US 2008/0281550 A1
(43) Pub. Date: Nov. 13, 2008

(54) SYSTEMS AND METHODS FOR CHARACTERIZING BALANCE FUNCTION

(75) Inventors: Richard Hogle, Madison, WI (US); Robert Beckman, Cross Plains, WI (US)

Correspondence Address: Casimir Jones, S.C., 440 Science Drive, Suite 203, Madison, WI, 53711, US

(73) Assignee: WICAB, INC., Middleton, WI (US)

(21) Appl. No.: 12/119,220

(22) Filed: May 12, 2008

Related U.S. Application Data
(60) Provisional application No. 60/928,881, filed on May 11, 2007.

(51) Int. Cl. G06F 15/00; A61B 5/00

(52) U.S. Cl. 702/127; 73/865.4

(57) ABSTRACT

The present invention relates to systems and methods for characterizing balance function. In particular, the present invention provides systems and methods for monitoring balance function (e.g., in a single individual and/or plurality of individuals), generating one or more databases comprising balance function data, processing and/or analyzing databases comprising balance function data, and characterizing balance function (e.g., in a single individual and/or plurality of individuals (e.g., utilizing databases comprising balance function data)). Systems and methods of the present invention find use in, among other things, research, diagnostic and therapeutic applications.

This utility patent application claims the benefit of and priority to U.S. Provisional Patent Application Ser. No. 60/928,881 filed May 11, 2007, hereby incorporated by reference in its entirety.

### FIELD OF THE INVENTION

The present invention relates to systems and methods for characterizing balance function. In particular, the present invention provides systems and methods for monitoring balance function (e.g., in a single individual and/or plurality of individuals), generating one or more databases comprising balance function data, processing and/or analyzing databases comprising balance function data, and characterizing balance function and assessing balance function for making diagnostic assessments (e.g., in a single individual and/or plurality of individuals (e.g., utilizing databases comprising balance function data)). Systems and methods of the present invention find use in, among other things, research, diagnostic and therapeutic applications.

### BACKGROUND OF THE INVENTION

There exist a wide variety of ailments, injuries, diseases and conditions that lead to one or more types of balance function defects (e.g. balance control, stability, etc.) in a subject. Such individuals often exhibit signs or symptoms of the balance function defect including a susceptibility to falling, swaying or other type of defect in body control (e.g., tremor), disorientation, the inability to walk straight and/or the inability to walk in crowds. Balance function defects may be the result of a wide variety of sensory and/or motor disorders that impair the posture and equilibrium control of the subject and may be indicative or symptomatic of underlying diseases.

A number of functional tests have been developed in order to assess balance control in a subject. These include dynamic gait index test, functional gait assessment test, Berg balance test, timed up and go test, and the dynamic task test. For each of these tests, an assessment of balance control in a subject is made by a physician or physical therapist monitoring a subject's performance throughout a number of motor tasks (e.g., such as standing, getting up out of a chair, walking, walking down steps, etc). The assessment comprises the physician and/or physical therapist providing the subject a score for how the physician and/or physical therapist believes the subject performed. Such methods are prone to subjective variables (e.g., empathy for the subject, administrator to administrator variability, personal traits, etc.) that may greatly influence the score received by a subject attempting to complete a test.

What is needed are better systems and methods for characterizing balance function in a subject.

### Definitions

To facilitate an understanding of the present invention, a number of terms and phrases are defined below:

As used herein, the term “subject” refers to a human or other vertebrate animal. It is intended that the term encompass patients.

As used herein, the term “amplifier” refers to a device that produces an electrical output that is a function of the corresponding electrical input parameter, and increases the magnitude of the input by means of energy drawn from an external source (i.e., it introduces gain). “Amplification” refers to the reproduction of an electrical signal by an electronic device, usually at an increased intensity. “Amplification means” refers to the use of an amplifier to amplify a signal. It is intended that the amplification means also includes means to process and/or filter the signal.

As used herein, the term “receiver” refers to the part of a system that converts transmitted waves into a desired form of output. The range of frequencies over which a receiver operates with a selected performance (i.e., a known level of sensitivity) is the “bandwidth” of the receiver.

As used herein, the term “transducer” refers to any device that converts a non-electrical parameter (e.g., sound, pressure or light), into electrical signals or vice versa.

As used herein, the terms “stimulator” and “actuator” are used herein to refer to components of a device that impart a stimulus (e.g., vibrotactile, electrotactile, thermal, etc.) to tissue of a subject. When referenced herein, the term stimulator provides an example of a transducer. Unless described to the contrary, embodiments described herein that utilize stimulators or actuators may also employ other forms of transducers.

The term “circuit” as used herein, refers to the complete path of an electric current.

As used herein, the term “resistor” refers to an electronic device that possesses resistance and is selected for this use. It is intended that the term encompass all types of resistors, including but not limited to, fixed-value or adjustable, carbon, wire-wound, and film resistors. The term “resistance” (R; ohm) refers to the tendency of a material to resist the passage of an electric current, and to convert electrical energy into heat energy.

The term “magnet” refers to a body (e.g., iron, steel or alloy) having the property of attracting iron and producing a magnetic field external to itself, and when freely suspended, of pointing to the magnetic poles of the Earth.

As used herein, the term “magnetic field” refers to the area surrounding a magnet in which magnetic forces may be detected.

As used herein, the term “electrode” refers to a conductor used to establish electrical contact with a nonmetallic part of a circuit, in particular, part of a biological system (e.g., human skin on tongue).

The term “housing” refers to the structure encasing or enclosing at least one component of a system and/or device of the present invention. In preferred embodiments, the “housing” is produced from a “biocompatible” material. In some embodiments, the housing comprises at least one hermetic feedthrough through which leads extend from the component inside the housing to a position outside the housing.

As used herein, the term “biocompatible” refers to any substance or compound that has minimal (i.e., no significant difference is seen compared to a control) to no irritant or immunological effect on the surrounding tissue. It is also intended that the term be applied in reference to the substances or compounds utilized in order to minimize or to avoid an immunologic reaction to the housing or other aspects of the invention. Particularly preferred biocompatible materials include, but are not limited to titanium, gold, platinum, sapphire, stainless steel, plastic, and ceramics.

As used herein, the term “implantable” refers to any device that may be implanted in a patient. It is intended that the term encompass various types of implants. In preferred embodiments, the device may be implanted under the skin (i.e., subcutaneous), or placed at any other location suited for the use of the device (e.g., within temporal bone, middle ear or inner ear). An implanted device is one that has been implanted within a subject, while a device that is “external” to the subject is not implanted within the subject (i.e., the device is located externally to the subject's skin).

As used herein, the term “hermetically sealed” refers to a device or object that is sealed in a manner that liquids or gases located outside the device are prevented from entering the interior of the device, to at least some degree. “Completely hermetically sealed” refers to a device or object that is sealed in a manner such that no detectable liquid or gas located outside the device enters the interior of the device. It is intended that the sealing be accomplished by a variety of means, including but not limited to mechanical, glue or sealants, etc. In particularly preferred embodiments, the hermetically sealed device is made so that it is completely leak-proof (i.e., no liquid or gas is allowed to enter the interior of the device at all).

As used herein the term “processor” refers to a device that is able to read a program from a computer memory (e.g., ROM or other computer memory) and perform a set of steps according to the program. Processor may include non-algorithmic signal processing components (e.g., for analog signal processing).

As used herein, the terms “computer memory,” “storage means,” “computer memory device,” and the like refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape, flash memory, and servers for streaming media over networks.

As used herein the terms “multimedia information” and “media information” are used interchangeably to refer to information (e.g., digitized and analog information) encoding or representing audio, video, and/or text. Multimedia information may further carry information not corresponding to audio or video. Multimedia information may be transmitted from one location or device to a second location or device by methods including, but not limited to, electrical, optical, and satellite transmission, and the like.

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein the term “security protocol” refers to an electronic security system (e.g., hardware and/or software) to limit access to processor, memory, etc. to specific users authorized to access the processor. For example, a security protocol may comprise a software program that locks out one or more functions of a processor until an appropriate password is entered.

As used herein the term “resource manager” refers to a system that optimizes the performance of a processor or another system. For example a resource manager may be configured to monitor the performance of a processor or software application and manage data and processor allocation, perform component failure recoveries, optimize the receipt and transmission of data, and the like. In some embodiments, the resource manager comprises a software program (e.g., incorporated into a system of the present invention).

As used herein the term “in electronic communication” refers to electrical devices (e.g., computers, processors, communications equipment) that are configured to communicate with one another through direct or indirect signaling. For example, a conference bridge that is connected to a processor through a cable or wire, such that information can pass between the conference bridge and the processor, are in electronic communication with one another. Likewise, a computer configured to transmit (e.g., through cables, wires, wireless signals, infrared signals, telephone lines, etc) information to another computer or device, is in electronic communication with the other computer or device.

As used herein the term “transmitting” refers to the movement of information (e.g., data) from one location to another (e.g., from one device to another) using any suitable means.

As used herein, the term “electrotactile” refers to a means whereby sensory channels (e.g., nerves) responsible for sensory functions are stimulated by an electric current. In some embodiments, the term refers to a means by which sensory channels (e.g., nerves) responsible for human touch (and/or taste) perception are stimulated by an electric current (applied via surface (or implanted) electrodes). The term electrotactile may be used interchangeably with the terms “electrocutaneous” and “electrodermal.”

As used herein, the term “balance” refers generally to factors related to a subject's ability to maintain a physical equilibrium and/or steadiness related to one or more objects. For example, “balance” may refer to a subject's ability to stand upright without displaying or feeling signs and/or symptoms of sway with regard to a vertical or horizontal axis (e.g., the ground or a visual target (e.g., a television or computer screen)). Thus, balance may refer to the stability produced by even distribution of weight on each side of a vertical axis (e.g., in a subject, this may occur by distributing weight evenly one's two feet (e.g., with eyes open or closed)).

As used herein, “balance function” refers generally to the ability of a subject to display balance under certain physical conditions (e.g., various physical tests that challenge a subject's ability to maintain balance under certain conditions). Accordingly, the term “balance function data” refers to any factual information (e.g., detected and/or acquired (e.g., through measurements, calculations and/or statistics) obtained by any means (e.g., an information sensing device (e.g., motion sensor (e.g., accelerometer))) that includes both useful and irrelevant or redundant information. Thus, “balance function data” may refer to raw data (e.g., collected by a motion sensor) as well as processed data (e.g., by software configured to analyze raw motion data).

### DETAILED DESCRIPTION OF THE INVENTION

The present invention relates to systems and methods for characterizing balance function and/or balance function data. In particular, the present invention provides systems and methods for monitoring balance function (e.g., in a single individual and/or plurality of individuals), generating one or more databases comprising balance function data, analyzing databases comprising balance function data, and characterizing balance function (e.g., in a single individual and/or plurality of individuals (e.g., utilizing databases comprising balance function data)). Systems and methods of the present invention find use in, among other things, research, diagnostic and therapeutic applications.

The vestibular system detects head movement by sensing head acceleration with specialized peripheral receptors in the inner ear that comprise semicircular canals and otolith organs. The vestibular system is important in virtually every aspect of daily life, because head acceleration information is essential for adequate behavior in three-dimensional space not only through vestibular reflexes that act constantly on somatic muscles and autonomic organs (see Wilson and Jones, Mammalian Vestibular Physiology, 2002, New York, Plenum), but also through various cognitive functions such as perception of self-movement (See, e.g., Buttner and Henn, Circularvection: psychophysics and single-unit recordings in the monkey, 374:274 (1981); Guedry et al., Aviat. Space Environ. Med., 50:205 (1979); Guedry et al., Aviat. Space Environ. Med., 52:304 (1981); Guedry et al., Brian Res. Bull., 47:475 (1998); Jell et al., Aviat. Space Environ. Med., 53:541 (1982); and Mergner et al., Patterns of vestibular and neck responses and their interaction: a comparison between cat cortical neurons and human psychophysics, 374:361 (1981)), spatial perception and memory (See, e.g., Berthoz et al., Spatial memory of body linear displacement: what is being stored? 269:95 (1995); Berthoz, The role of inhibition in the hierarchical gating of executed and imagined movements, 3:101 (1996); Bloomberg et al., Vestibular-contingent voluntary saccades based on cognitive estimates of remembered vestibular information, 41:71 (1988); and Nakamura and Bronstein, The perception of head and neck angular displacement in normal and labyrinthine-defective subjects. A quantitative study using a ‘remembered saccade’ technique, 188:1157 (1995)), visual spatial constancy (See, e.g., Anderson, Exp. Psychol. Hum. Percept. Perform., 15:363 (1989) and Bishop, Stereopsis and fusion, 26:17 (1974)), visual object motion perception (See, e.g., Mergner, Role of vestibular and neck inputs for the perception of object motion in space, 89:655 (1992) and Mesland, Object motion perception during ego-motion: patients with a complete loss of vestibular function vs. normals, 40:459 (1996)), and even locomotor navigation (See, e.g., Wiener, Spatial and behavioral correlates of striatal neurons in rats performing a self-initiated navigation task, 13:3802 (1993)). Vestibular input functions also include: egocentric sense of orientation, coordinate system, internal reference center, muscular tonus control, and body segment alignment (See, e.g., Honrubia and Greenfield, A novel psychophysical illusion resulting from interaction between horizonal vestibular and vertical pursuit stimulation, 19:513 (1998)).

Subjects with defective vestibular systems (e.g., due to damage to the vestibular system (e.g., such as from an adverse reaction to antibiotic medications, traumatic brain injury, stroke, Meniere's disease, or for any other reason)) as well as subjects with defects (e.g., due to injury, stroke, or disease, etc.) in non-vestibular physiological systems related to balance function (e.g., of proprioceptive, central nervous system (CNS) and/or non-physiologic origin) experience functional difficulties related to balance. For example, subjects with defective balance function exhibit and/or suffer from many types of signs and symptoms of balance functional defects (e.g., compared to a cohort of “healthy” individuals that do not exhibit or suffer from any such signs and symptoms) including, but not limited to, dizziness, postural “wobbling” (e.g., during both sitting and standing positions), unstable gait, inability to walk straight and/or upright, tremor or other type of uncontrollable shaking, disorientation, swaying, falling, and/or others signs and/or symptoms (e.g., that make it difficult or impossible, for example, to walk straight, at the same speed, up or down stairs or in the dark (e.g., without risk of falling)). These signs and symptoms can precipitate multiple problems with posture control, movement (e.g., up-down, forward-backward motion in space including unsteady gait) and various balance-related difficulties, like oscillopsia (See, e.g., Baloh, Changes in the human vestibulo-occular reflex after loss of peripheral sensitivity, 16:222 (1991)). Unsteady gait can be especially evident at night (or in persons with low visual acuity). Loss of balance functions can be particularly incapacitating for elderly persons.

Oscillopsia (e.g., due to the loss of vestibulo-ocular reflexes) can be a distressing illusory oscillation of the visual scene (See, e.g., Brant, Man in motion. Historical and clinical aspects of vestibular function. A review. 114:2159 (1991)). When walking, subjects are unable to fixate on objects because the surroundings are bounding up and down. In order to see the faces of passerbies, subjects learn to stop and hold their heads still. When reading, such patients learn to place their hand on their chin to prevent slight movements associated with pulsation of blood flow.

During development of certain embodiments of the present invention, certain objectively characterizable parameters were identified in subjects (e.g., with loss of balance function (e.g., due to vestibular system defects or other types of balance system defects (e.g., proprioceptive, central nervous system (CNS), or non-physiologic origin))) and determined to be diagnostically informative. These parameters include, but are not limited to, mean-position drift, sway, and periodic large-amplitude perturbations.

Accordingly, in some embodiments, in contrast to currently utilized tests for scoring and assessing balance function in a subject (e.g., that are prone to subjective input from a test administrator), systems and methods of the present invention provide objective, quantifiable assessment of balance control and/or function (e.g., in some embodiments, via monitoring, storing, processing and assessing balance function data (e.g., mean-position drift, sway, and/or periodic large-amplitude perturbations)). In some embodiments, accumulation of objective balance function data (e.g., via monitoring mean-position drift, sway, and/or periodic large-amplitude perturbations) may be utilized to generate databases of balance function data. In some embodiments, the databases of balance function data can in turn be utilized (e.g., processed) by a system of the present invention to provide objective scoring and assessment of a user (e.g., assessment of a user's balance (e.g., balance control and/or function) and/or assessment of a user's health status (e.g., for diagnosing the presence of a disease or condition)).

For example, in some embodiments, systems and methods of the present invention are used diagnostically (e.g., to predict and/or monitor the onset, progression and/or regression of signs and/or symptoms (e.g., of disease or other health conditions (e.g., stroke)) or to otherwise monitor performance (e.g., during therapy, rehabilitation, training, etc. (e.g., of patients, the elderly and/or athletes)). In some embodiments, systems and methods of the present invention are utilized to monitor a subject's proficiency (e.g., balance control and/or function) in one or more physical tests (e.g., dynamic gait index test, functional gait assessment test, Berg balance test, timed up and go test, and the dynamic task test) and/or to compare a subject's results to a database of “normal”/“healthy” and “non-normal”/“non-healthy” results (e.g., to provide an objective score and/or assessment of the subject being tested). In some embodiments, systems and methods of the present invention, comparing a subject's performance data to balance function data stored on a computer, can assess and provide information regarding staging of a disease (e.g., Parkinson's disease, Meniere's disease, muscular dystrophy, or other type of disease comprising a balance function defect), confirmation of an event (e.g., that a stroke occurred), stage of training (e.g., for an athletic event, during rehabilitation from stroke, traumatic brain injury, etc.), or other types of information related to balance control and/or function in a subject.

In some embodiments, subjects taking gentamycin can be monitored for loss of balance and/or damage to vestibular function thereby permitting a physician to discontinue or alter treatment so as to prevent or reduce unwanted side effects of the drug. In such embodiments, head displacement as a function of body position may be monitored and compared to a normal baseline or to look for variation in a particular subject over time. In some embodiments, because posture and balance deteriorate with age, the system may also be used as a biomarker of biological age of a subject. Diagnostic methods utilizing the systems and methods of the present invention may be used as an initial screening method for a subject or may be used to monitor status during or after some treatment course of action.

The present invention is not limited by the nature of the sensors, methods and/or devices utilized to monitor and/or accumulate balance function data. Examples include use of various accelerometers, MEMS technology, and positional sensors that are described more fully herein.

In some embodiments, systems and methods of the present invention are utilized to provide objective, diagnostic assessment and/or rehabilitation of balance function by placing a subject through one or more physical tests (e.g., commonly used to subjectively measure balance function (e.g., dynamic gait index test, functional gait assessment test, Berg balance test, timed up and go test, and the dynamic task test)), in conjunction with the subject wearing one or more means of detecting and transducing balance function data. In some embodiments, the balance function data is transferred (e.g., wirelessly, via cable, etc.) from the detecting means to a data storage device. In some embodiments, the balance function data of a plurality of users is accumulated and processed to generate a “balance function footprint” (e.g., representing a particular type of sub-population of subjects (e.g., “healthy” subjects versus “non-healthy” subjects (e.g., that display or suffer from one or more signs or symptoms of a balance function defect (e.g., due to vestibular, proprioceptive, central nervous system (CNS), and/or non-physiologic defects (e.g., resulting from traumatic brain injury, stroke, and/or disease (e.g., Miniere's disease, Parkinson's disease, muscular dystrophy, etc.))))). Thus, as used herein, the term “balance function footprint” refers to balance function data (e.g., a trace pattern of balance function data) that is representative of (e.g., derived and/or standardized from) a class of individuals that display signs and symptoms of a particular etiology (e.g., of a disease, disorder, or condition of health). In some embodiments, a system of the present invention comprises processing means (e.g., software, processor capable or running the software, memory, etc.) that compares balance function data acquired from a subject to one or more balance function footprints stored on a storage means of the system. In some embodiments, processing means and/or software of a system of the present invention provides a score and/or assessment of the subject based upon the comparison between the subject's balance function data and one or more balance function footprints.

The present invention is not limited by the number of balance function footprints that can be generated and/or stored on a system of the present invention. Accordingly, in some embodiments, a subject's balance function data is compared (e.g., using software) to balance function data (e.g., balance function footprints) previously stored on a data storage device of the system so as to provide a characterization/assessment of the balance function of the subject (e.g., as being normal and/or healthy or being correlated with balance function data (e.g., a balance function footprint) of one or more “non-healthy” subjects (e.g., suffering from a disease or disorder).

In some embodiments, balance function data generated from monitoring a subject (e.g., using one or more sensors and/or types of sensors to monitor balance function data (e.g., mean-position drift, sway, and/or periodic large-amplitude perturbations) of a subject executing one or more types of tests (e.g., dynamic gait index test, functional gait assessment test, Berg balance test, timed up and go test, and the dynamic task test)) can be processed and compared to one or more balance function footprints (e.g., generated using the systems and methods of the present invention) to generate (e.g., using a processor and software of a system of the present invention) an objective assessment/characterization of a subject. Such an assessment may be used for the diagnosis and/or monitoring of progression and/or regression of balance function. Thus, in some embodiments, the present invention provides systems and methods for automatic diagnosis of a balance function defect (e.g., due to a vestibular, a proprioceptive, a CNS lesion, or a non-physiologic balance disorder) or of optimization (e.g., fine-tuning) of healthy balance function.

In some embodiments, a system of the present invention provides actual balance function data of a subject. In some embodiments, a system of the present invention provides balance function data encompassing a range of values from healthy to non-healthy subjects (e.g., data from balance function defective individuals). Thus, in some embodiments, raw data can be reviewed and analyzed by a user of the systems and methods of the present invention instead of or in conjunction with using software configured to analyze and/or assess subject balance function data. In some embodiments, a user can manually determine (e.g., using raw data) or can use software configured to determine if a subject's balance function has improved, deteriorated or remained the same over a period of time (e.g., a day, two days, a week, a month, three months, six months, nine months, a year, two years or more).

In some embodiments, systems and methods of the present invention are utilized for both objective, diagnostic assessment as well as for rehabilitation. For example, in some embodiments, systems and methods of the present invention can be used with one or more devices, systems and/or methods described in U.S. patent application Ser. Nos. 10/998,222, 11/033,246, 11/234,635, 11/234,457, 11/234,453, and 11/657,376, each of which is hereby incorporated by reference in its entirety. Thus, in some embodiments, systems and methods of the present invention can be utilized with electrotactile stimulation of the tongue to provide rehabilitative information to a subject. In some embodiments, data generated during rehabilitation is utilized to monitor a subject's balance function over time (e.g., balance function improvement or deterioration). It is contemplated that such rehabilitative information can provide a subject balance function information that otherwise is lacking and/or that is not understood by a subject thereby providing a subject an immediate benefit (e.g., improved balance function (e.g., improved gait and/or decreased dizziness and/or body sway)) from the diagnostic methods described herein. However, the present invention is not limited by electrotactile stimulation of the tongue to provide beneficial information to a subject. Indeed, any information that can be accumulated, processed and presented to a user using the systems and methods of the present invention is contemplated to be information that can be presented to a subject. For example, identification of balance function data of a subject that correlates with one or more balance function footprints in and of itself may provide beneficial information to a user of the systems and methods of the present invention (e.g., this information may allow a user to address one or more issues related to balance function (e.g., that a subject was unaware needed addressing)).

The present invention is not limited by the means of detecting balance function data nor the type of balance function data detected, stored, processed and/or assessed. In some embodiments, balance function data detected, stored, processed and/or assessed comprises any type of objectively characterizable parameters associated with balance function (e.g., loss or enhancement of balance function due to vestibular system defects or other types of balance system defects (e.g., proprioceptive, central nervous system (CNS), or non-physiologic origin))) including, but not limited to, mean-position drift, sway, periodic large-amplitude perturbations, and any type of measurable motion and/or movement of a subject. In some embodiments, means of detecting balance function data comprise one or more different types of sensors attached to one or more different parts of a subject's body. For example, in some embodiments, one or more sensors are attached to a subject so as to detect/monitor motion and/or movement (e.g., acceleration, velocity, turning (e.g., about an axis (e.g., vertical and/or horizontal axis)), roll, pitch, angular velocity, etc.) of a subject's head. In some embodiments, one or more sensors are attached to a subject so as to detect/monitor motion and/or movement (e.g., acceleration, velocity, turning (e.g., about an axis (e.g., vertical and/or horizontal axis)), roll, pitch, angular velocity, etc.) of a subject's upper torso. In some embodiments, one or more sensors are attached to a subject so as to detect/monitor motion and/or movement (e.g., acceleration, velocity, turning (e.g., about an axis (e.g., vertical and/or horizontal axis)), roll, pitch, angular velocity, etc.) of a subject's head and upper torso. In some embodiments, one or more sensors are attached to a subject so as to detect/monitor motion and/or movement (e.g., acceleration, velocity, turning (e.g., about an axis (e.g., vertical and/or horizontal axis)), roll, pitch, angular velocity, etc.) of a subject's waist. In some embodiments, one or more sensors are attached to a subject so as to detect/monitor motion and/or movement (e.g., acceleration, velocity, turning (e.g., about an axis (e.g., vertical and/or horizontal axis)), roll, pitch, angular velocity, etc.) of a subject's left and/or right shoulders, or one or more combination of two or more of each of the body parts described above, or other body parts.

In some embodiments, the location of the one or more sensors on the subject's body is determined by the axis of sensitivity of the sensors. For example, one or more sensors (e.g., mounted on the front or back of the subject's torso) may be used to detect roll angular deviations of the patient's torso from the vertical, and roll angular velocities. A second sensor mounted on the side of the subject's torso (e.g., under an arm) may be used to detect pitch angular deviations from the vertical, and pitch angular velocities. In some embodiments, additional sensors can be utilized that register yaw (i.e., turning about the vertical axis, angular deviations, and yaw angular velocities (e.g., of the head, torso, waist, or any combination thereof)).

In some embodiments, a single sensor is utilized to detect motion and/or movement (e.g., acceleration, velocity, turning (e.g., about an axis (e.g., vertical and/or horizontal axis)), roll, pitch, angular velocity, etc.) from a single location of a subject. In some embodiments, this location is the head. The present invention is not limited by a placement location on the head of a user. Indeed, a variety of locations on the head of a user are contemplated to be useful for monitoring motion and/or movement of a subject's head including, but not limited to, the forehead or other anterior location, posterior head placement, above the left or right ear and other locations (e.g., that do not interfere with a subjects line of sight). In some embodiments, the single location is the chest or the back, the swell of the back, on or around the bellybutton, or other locations (e.g., that do not restrict movement in any significant manner).

In some embodiments, one or more groups of sensors can be utilized to measure motion and/or movement through one or more planes of the subject with respect to the gravitational plane. For example, one group of sensors (e.g., three or more sensors) can be configured such that one sensor is placed centered on a subject's forehead with two other sensors being placed such that a sensor is present on each of the user's shoulders. Thus, this combination of sensors may define a first surface plane of the subject (e.g., that dissects the subject at or around the shoulders and/or forehead). In some embodiments, a second group of sensors (e.g., three or more sensors) can be placed on the subject such that one sensor is placed on each the left side and right side of the waist as well as one on the back of the neck. This may define a second surface plane of the subject's body. Thus, in some embodiments, the present invention provides detecting motion, velocity, acceleration and/or any type of movement through one or both of these planes. Thus, balance function data (e.g., representing each or all of mean-position drift, sway, and periodic large-amplitude perturbations) can be detected, stored, processed and/or analyzed by the systems and methods of the present invention. It should be noted, however, that the present invention is not limited by the sensor configurations described above. Indeed, any configuration of sensors capable of providing information regarding balance function of a subject are contemplated to be useful in the present invention. For example, a subject may wear one or more sensors at one or more positions of other locations of the body including, but not limited to, the legs, arms, hands, elbows, knees, neck, head, feet and/or combinations thereof. It is contemplated that a configuration of one or more sensors described herein are capable of acquiring various types of balance function data from a subject that is performing one of more tasks associated with one or more tests (e.g., dynamic gait index test, functional gait assessment test, Berg balance test, timed up and go test, and the dynamic task test (e.g., such that all balance function data necessary to score and/or assess a subject or for generating balance function footprints from a plurality of users is acquired)).

In some embodiments, a system of the present invention comprises software and/or other means for processing balance function data detected by the one or more sensors described herein. In some embodiments, the software and/or other means for processing balance function data generate balance function footprints from the balance function data. In some embodiments, the software and/or other means for processing balance function data calculate and/or extrapolate information related to a subject's movement (e.g., motion, velocity, acceleration, turning about the vertical axis, vertical and roll angular velocities, roll angular deviations, vertical angular deviations, pitch angular velocities, pitch angular deviations, angular velocities and/or any type of movement). In some embodiments, the software and/or other means for processing balance function data compare balance function data acquired from one or a plurality of sensors from one or a plurality of users to one or a plurality of balance function footprints thereby generating a score and/or assessment of the subject.

In some embodiments, a system of the present invention further comprises a resource manager. In some embodiments, the resource manager is configured to run various types of software (e.g., data collection software, data storage software, data comparison and/or analysis software, system software (e.g., maintenance software) and/or other types of software) present in a system of the present invention. In some embodiments, the resource manager is configured to monitor the performance of components of a system (e.g., processor and/or one or more motion sensor) and/or communication between various components (e.g., processor and one or more sensors) of the system. In some embodiments, the resource manager is configured to optimize receipt and transmission data. In some embodiments, the resource manager is software or other type of code present on a system of the present invention.

The present invention is not limited by the manner in which sensors are attached to and/or affixed to a subject. In some embodiments, sensors are attached using VELCRO, elastic straps, bands (e.g., head-band, wrist-band, waist-band, chest-band, etc.), tape, or any other means well known to those of skill in the art for a snug, non-moveable, optionally removable attachment to a subject. In some embodiments, sensors are attached against the skin. In some embodiments, sensors are attached over a subject's garments. In some embodiments, sensors are implantable (e.g., removably or non-removably implantable) in a subject.

The present invention is not limited by the type of sensors utilized in the systems and methods disclosed herein. In some preferred embodiments, the sensor utilized is capable of providing a direct measurement of force and/or movement (e.g., motion, velocity, acceleration, turning about the vertical axis, vertical and roll angular velocities, pitch angular velocities, angular velocities and/or any type of movement). For example, in some embodiments, a sensor detects and/or measures angular velocity. In some embodiments, the sensor is substantially insensitive to the gravity vector and/or to linear accelerations (e.g., straight up and down, backward and forward, and side-to-side motions (e.g., of the subject's entire body). In some embodiments, a sensor comprises an accelerometer. For example, in some embodiments, the sensor is a miniature 2-axis accelerometer (e.g., Analog Devices ADXL202). In some embodiments, the accelerometer detects and/or measures anterior-posterior and medial-lateral angular displacement data.

In some embodiments, the accelerometer is nominally oriented in the horizontal plane. In this position, it normally senses both rotation and translation. However, given the nature of a particular task (e.g., quiet upright sitting and/or one or more tasks assigned in a dynamic gait index test, a functional gait assessment test, a Berg balance test, a timed up and go test, and/or a dynamic task test), all non-zero acceleration data can be recorded in both the x- and y-axis (the M/L and A/P direction, respectively), and can be ascribed to angular displacement or tilt of the head and not translation. In some embodiments, after instructing a subject to assume any particular test position or movement, an initial value of the sensor can be recorded (e.g. at the start of each trail within a test) and subsequently used as the zero-reference. In some embodiments, using a small angle approximation, and given that the sensor output is proportional to the angular displacement from the zero position, the instantaneous angle can be calculated as:

Θₓ=sin⁻¹ aₓ/g (Eq. 1)

Θ_{y}=sin⁻¹ a_{y}/g (Eq. 2)

where g is the gravity vector and both aₓ and a_{y} are the vector components in the respective axis.

In some embodiments, a sensor utilized is the LITEF MICRO FORS 36 Fiber Optic Rate Sensor, made by LITEF GmbH of Freiburg, Germany, D-79007. This sensor is an angular velocity sensor that may be programmed to provide either angular deviation or angular velocity information in digital form, at a selected scale factor, to a processor of a system of the present invention that can in turn command storage and/or analylsis of balance function data acquired (e.g., digital angular deviation or velocity values processed and/or characterized into usable information (e.g., a subject score and/or assessment)).

In some embodiments, sensors that measure Coriolis forces in vibrating structures to sense angular velocities may also be used.

In some embodiments, microelectromechanical systems (MEMS) sensors are utilized. Examples of such sensors include, but are not limited to, MEMS linear acceleration transducers (accelerometers) and MEMS gyroscopes. One of skill in the art knows well that there are many manufactures of such products and that these devices are readily available.

In some embodiments, balance function data sensors are configured using pairs of accelerometers set at fixed distances from one another on the subject's body. Such devices may be used to measure angular accelerations, which may then be transformed into angular velocity and angular deviation values by suitable analog or digital integration algorithms implemented, for example, by a processor of a system of the present invention.

In some embodiments, the present invention provides systems and methods for objectively characterizing balance function in a subject thereby eliminating subjectivity present in current testing methods (e.g., that may alter subject performance scores and assessments). In some embodiments, systems and methods of using the same are relatively inexpensive and capable of monitoring, storing, processing and assessing balance function data from one or a plurality of body locations from one or a plurality of subjects.

In some embodiments, balance function data is acquired as angular velocity signals. In some embodiments, balance function data is acquired as angular displacement signals. In some embodiments, a processor of the present invention converts angular displacement signals into angular velocity data. In some embodiments, balance function data acquired as angular acceleration signals provided to a sensor is transferred to a processor and converted and/or processed by the processor into angular velocity information and/or displacement information. It is contemplated that standard digital differentiation and integration algorithms may be used by a system processor and/or resource manager and/or software to perform such tasks (e.g., differentiation and integration functions), as necessary.

In some embodiments, the present invention provides a system comprising sensors configured to acquire balance function data of a subject, wherein the sensors further are configured to transfer and/or transducer balance function data to a data storage device in communication with a processor, wherein the processor (e.g., running software) comprises the ability to analyze the balance function data and provide a score and/or assessment of the subject. In some embodiments, the system further comprises a subject, wherein the subject is tasked with performing one or more physical acts that are part of a test procedure (e.g., a dynamic gait index test, a functional gait assessment test, a Berg balance test, a timed up and go test, and a dynamic task test). Thus, a system of the present invention can generate a data set (e.g., comprising balance function data) comprising a subject's entire performance on a test (e.g., comprising individual testing steps that are part of each test). Examples of testing steps (e.g., for which balance function data can be acquired, stored, processed and/or assessed using a system of the present invention) include, but are not limited to, walking, walking fast or slow, changing speeds, head turning, looking up, looking down, body turning, moving around and/or over objects, walking up and/or down stairs, pivot turning, entering a designated space, walking while head turning, walking on a narrow space, walking and/or standing with eyes open and/or closed, sitting down and standing up to/from a seated position, picking up objects, placing one or both feet in a designated spot, standing on one foot or leg, and/or rising from a prostrate position (See, e.g., Example 1).

Similarly, the present invention is not limited by the type of subject that uses and/or that is monitored and/or diagnosed by the systems and methods of the present invention. Indeed, any type of subject that desires an objective assessment of balance function will find the systems and methods of the present invention useful. Such subject may include, but are not limited to, the elderly, subjects with signs and/or symptoms of disease (e.g., diseases known to be associated with loss of balance function (e.g., Meniere's disease, Parkinson's disease, autoimmune diseases) as well as diseases not traditionally associated with loss of balance function), injury (e.g., traumatic brain injury), stroke, addiction to a substance (e.g., drug and/or alcohol), subjects described in U.S. patent application Ser. Nos. 10/998,222, 11/033,246, 11/234,635, 11/234,457, 11/234,453, and 11/657,376 (each of which is hereby incorporated by reference in its entirety) and healthy subjects (e.g., training for an athletic event or simply trying to improve and/or refine balance).

In some embodiments, systems and methods of the present invention overcome the subjective input associated with various balance tests (dynamic gait index test, functional gait assessment test, Berg balance test, timed up and go test, and the dynamic task test) by providing objective, non-human processed and/or scored and/or assessed results. In some embodiments, a system of the present invention is compact and portable and can be used by anyone with or without the presence of a health professional. In some embodiments, such a system permits a subject to enjoy daily assessment of balance function (e.g., regardless of when and where the user is located).

For example, in some embodiments, the present invention provides a system for acquiring, storing, processing and/or assessing balance function comprising a portable unit (e.g., that can be worn on a subject's body) comprising a processor, storage means and processing means, and one or a plurality of sensors (e.g., motion sensors described herein) that acquire and transfer and/or transducer balance function data (e.g., to the processor and/or storage means). In some embodiments, the portable unit comprises software capable of performing analysis and diagnosis of balance function (e.g., based on balance function footprints stored on the storage means of the unit). In some embodiments, the unit is capable of sending (e.g., wirelessly, via a cable, or other means known in the art) analysis and diagnostic information to a computer (e.g., handheld computer, desktop or laptop computer) and/or a printer. In some embodiments, the system further comprises a monitor that communicates with the unit such that information stored (e.g., instructions for performing a test or task) or processed (e.g., subject assessment) on the unit can be presented on the monitor. In some embodiments, portions of the system can be worn on a subject's body (e.g., motion sensors) whereas other portions of the system (e.g., processor, storage means, software, etc.) are located in housing placed somewhere other than the subject's body. In some embodiments, components of a system of the present invention communicate via wireless means. Wireless means are well known to those of skill in the art and include, but are not limited to, BLUETOOTH technology, infra-red signals, 802.11 signals (e.g., a, b, g, e signals) and other types of wireless signals (e.g., digital and/or analog signals). In some embodiments, a subject is located in a particular spot (e.g., the subject's home) wherein several components of system are also located (e.g., motion sensors and processor), whereas other components are located at a different, remote location (e.g., storage means, processor, resource manager). Thus, in such a system, balance function data may be communicated to other components of the system (e.g., via the Internet), wherein when the balance function data is received, it is processed and/or analyzed (e.g., to provide a score and/or assessment and/or to generate a balance function footprint), with or without feedback (e.g., score and/or assessment) provided to the subject.

In some embodiments, in order to evaluate how a subject performs one or more physical tests (e.g., dynamic gait index test, functional gait assessment test, Berg balance test, timed up and go test, and the dynamic task test) over time, specific tasks (e.g., that constitute a portion or the entire test) can be measured on multiple days. In some embodiments, a subject that uses a method of the present invention may also record other conditions that the subject experienced that day (e.g., a fall, dizziness, tiredness due to poor sleep the night before, or comments regarding other conditions of the subject). In some embodiments, it is preferred that each test that is monitored for balance function data be repeated in the same or nearly identical setting (e.g., a quiet, well lit room). Thus, using such methods, it is possible to standardize a subjects balance function data (e.g., and then use the standardized data for comparison to balance function footprints).

In some embodiments, in addition to providing an accurate, objective scoring and/or assessment of a subject, and/or rehabilitation, systems and methods of the present invention may be used to monitor the status of one or a plurality of individuals (e.g., that are at risk of falling (e.g., in homes, hospitals, nursing homes, etc.). Furthermore, a system of the present invention can be used to provide a complete record of movement (e.g., body movement (e.g. a specific part of a body's movement (e.g., head movement))). For example, a system of the present invention can monitor falls, near falls, unsteadiness, sway, or other type of motion and/or movement described herein and generate an objective record of the same. In some embodiments, a system of the present invention is integrated with in home medical alert systems to provide information to a central monitoring location that a subject has fallen, is having a seizure, or is experiencing some other type of event. Accordingly, a system of the present invention can be utilized to monitor a very large number of individuals simultaneously (e.g., by a remote system processor and/or resource manager and/or other type of software). As described herein, system components of the present invention can be configured in such a way so as to have very little to no interference with the day to day activities of a user of a system of the present invention.

Thus, in some embodiments, the functions performed by a system of the present invention may be separated and tasked to more than one processor (e.g., several microprocessor based systems at different locations) each of which is in communication with each other (e.g., monitored by one or more resource managers).

It should be understood that while certain embodiments of the present invention have been described, the present invention is not confined to or limited to such embodiments, implementations and/or applications described herein, but rather the claims appended hereto are intended to cover all such embodiments, implementations and/or applications that fall within the true spirit and scope of the present invention.

### Experimental

The following examples are provided in order to demonstrate and further illustrate certain preferred embodiments and aspects of the present invention and are not to be construed as limiting the scope thereof.

### EXAMPLE 1

### Balance Function Tests

A system (e.g., comprising sensors capable of acquiring balance function data of a subject, wherein the sensors further are capable of transferring and/or transducing balance function data to a data storage device in communication with a processor, wherein the processor (e.g., running software) comprises the ability to analyze the balance function data and provide a score and/or assessment of the subject) of the present invention finds use in detecting motion and/or movement during a variety of physical tasks. Several physical test have been developed to score and/or assess balance function. As described herein, these test suffer from subjective input from those responsible for scoring and/or assessing performance.

Thus, a system of the present invention can be utilized to objectively acquire, store, process and analyze/assess subject performance in various physical tests. Several physical tests find use with the present invention including, but not limited to, dynamic gait index test, functional gait assessment test, Berg balance test, timed up and go test, and the dynamic task test.

### Dynamic Gait Index

Description:
Developed to assess the likelihood of falling in older adults.
This scale was designed to test eight facets of gait.
Equipment needed: Box (Shoebox)
   Cones (2)
   Stairs
Completion Time: 15 minutes
Scoring: A four-point ordinal scale, ranging from 0-3. “0” indicates the lowest level of function and “3” the highest level of function.
   Total Score=24
Interpretation: <19=predictive of falls in the elderly
   >22=safe ambulators

### Dynamic Gait Index

### Gait Level Surface ______

Instructions: Walk at your normal speed from here to the next mark (20′).
Grading: Mark the lowest category that applies.
(3) Normal: Walks 20′, no assistive devices, good speed, no evidence for imbalance, normal gait pattern.
(2) Mild Impairment: Walks 20′, uses assistive device, slower speed, mild gait deviations.
(1) Moderate Impairment: Walks 20′, slow speed, abnormal gait pattern, evidence for imbalance.
(0) Severe Impairment: Cannot walk 20′ without assistance, severe gait deviations or imbalance.

### Change in Gait Speed ______

Instructions: Begin walking at your normal pace (for 5′), when I tell you “go,” walk as fast as you can (for 5′). When I tell you “slow,” walk as slowly as you can (for 5′).
Grading: Mark the lowest category that applies.
(3) Normal: Able to smoothly change walking speed without loss of balance or gait deviation. Shows a significant difference in walking speeds between normal, fast and slow speeds.
(2) Mild Impairment: Is able to change speed but demonstrates mild gait deviations, or not gait deviations but unable to achieve a significant change in velocity, or uses an assistive device.
(1) Moderate Impairment: Makes only minor adjustments to walking speed, or accomplishes a change in speed with significant gait deviations, or changes speed but has a significant gait deviation, or changes speed but loses balance but is able to recover and continue walking.
(0) Severe Impairment: Cannot change speeds, or loses balance and has to reach for wall or be caught.
   Gait with Horizontal Head Turns ______
Instructions: Begin walking at your normal pace. When I tell you to “look right,” keep walking straight, but turn your head to the right. Keep looking to the right until I tell you “look left,” then keep walking straight and turn your head to the left. Keep your head to the left until I tell you “look straight,” then keep walking straight, but return your head to the center.
Grading: Mark the lowest category that applies.
(3) Normal: Performs head turns smoothly with no change in gait.
(2) Mild Impairment: Performs head turns smoothly with slight change in gait velocity, i.e., minor disruption to smooth gait path or uses walking aid.
(1) Moderate Impairment: Performs head turns with moderate change in gait velocity, slows down, staggers but recovers, can continue to walk.
(0) Severe Impairment: Performs task with severe disruption of gait, i.e., staggers outside 15″ path, loses balance, stops, reaches for wall.
   Gait with Vertical Head Turns ______
Instructions: Begin walking at your normal pace. When I tell you to “look up,” keep walking straight, but tip your head up. Keep looking up until I tell you “look down,” then keep walking straight and tip your head down. Keep your head down until I tell you “look straight,” then keep walking straight, but return your head to the center.
Grading: Mark the lowest category that applies.
(3) Normal: Performs head turns smoothly with no change in gait.
(2) Mild Impairment: Performs head turns smoothly with slight change in gait velocity, i.e., minor disruption to smooth gait path or uses walking aid.
(1) Moderate Impairment: Performs head turns with moderate change in gait velocity, slows down, staggers but recovers, can continue to walk.
(0) Severe Impairment: Performs task with severe disruption of gait, i.e., staggers outside 15″ path, loses balance, stops, reaches for wall.

### Gait and Pivot Turn ______

Instructions: Begin walking at your normal pace. When I tell you “turn and stop,” turn as quickly as you can to face the opposite direction and stop.
Grading: Mark the lowest category that applies.
(3) Normal: Pivot turns safely within 3 seconds and stops quickly with no loss of balance.
(2) Mild Impairment: Pivot turns safely in >3 seconds and stops with no loss of balance.
(1) Moderate Impairment: Turns slowly, requires verbal cueing, requires several small steps to catch balance following turn and stop.
(0) Severe Impairment: Cannot turn safely, requires assistance to turn and stop.

### Step Over Obstacle ______

Instructions: Begin walking at your normal speed. When you come to the shoebox, step over it, not around it, and keep walking.
Grading: Mark the lowest category that applies.
(3) Normal: Is able to step over the box without changing gait speed, no evidence of imbalance.
(2) Mild Impairment: Is able to step over box, but must slow down and adjust steps to clear box safely.
(1) Moderate Impairment: Is able to step over box but must stop, then step over. May require verbal cueing.
(0) Severe Impairment: Cannot perform without assistance.

### Step Around Obstacles ______

Instructions: Begin walking at normal speed. When you come to the first cone (about 6′ away), walk around the right side of it. When you come to the second cone (6′ past first cone), walk around it to the left.
Grading: Mark the lowest category that applies.
(3) Normal: Is able to walk around cones safely without changing gait speed; no evidence of imbalance.
(2) Mild Impairment: Is able to step around both cones, but must slow down and adjust steps to clear cones.
(1) Moderate Impairment: Is able to clear cones but must significantly slow, speed to accomplish task, or requires verbal cueing.
(0) Severe Impairment: Unable to clear cones, walks into one or both cones, or requires physical assistance.

### Steps ______

Instructions: Walk up these stairs as you would at home, i.e., use the railing if necessary. At the top, turn around and walk down.
Grading: Mark the lowest category that applies.
(3) Normal: Alternating feet, no rail.
(2) Mild Impairment: Alternating feet, must use rail.
(1) Moderate Impairment: Two feet to a stair, must use rail.
(0) Severe Impairment: Cannot do safely.
TOTAL SCORE: ______ /24

### Clinical Significance

The Dynamic Gait Index (DGI) is an evaluative tool used by therapists designed to measure a patient's ability to perform movement tasks while walking and to determine their likelihood of falling. It rates performance from 0 (poor) to 3 (excellent) on eight different gait tasks, including gait on even surfaces, gait when changing speeds, gait and head turns in a vertical or horizontal direction, stepping over or around obstacles, and gait with pivot turns and steps. Scores on the Dynamic Gait Index range from 0 to 24. A follow up DGI performed after a course of rehabilitation treatment is an indicator of efficacy of treatment.

Shumway-Cook A, Woollacott M. Motor Control Theory and Applications, Lippincott Williams and Wilkins, Baltimore, 2001:401-406.

Shumway-Cook A, Gruber W, Baldwin M, Liao S. “The effect of multidimensional exercises on balance, mobility, and fall risk in community-dwelling older adults.” Physical Therapy. Washington: January 1997. Vol 77, Iss. 1:46-58.

Shumway-Cook A, Baldwin M Pollisar N, Gruber W. “Predicting the probability of falls in community dwelling older adults.” Physical Therapy 1997a; 77:812-819.

### Functional Gait Assessment

Description: Developed to assess ability during higher level gait tasks in adults with vestibular disorder.
   This scale was designed to test ten facets of gait.
Equipment needed: 2 Boxes (Shoebox)
   Stairs
   Stopwatch
   A marked 6-m (20-ft) walkway that is marked with a 30.48-cm (12-in) width.
Completion Time: 20 minutes
Scoring: A four-point ordinal scale, ranging from 0-3. “0” indicates the lowest level of function and “3” the highest level of function.
   Total Score=30
Interpretation: No published norms at this time.

### Functional Gait Assessment

### ______1. Gait Level Surface

Instructions: Walk at your normal speed from here to the next mark (6 m [20 ft]).
Grading: Mark the highest category that applies.
(3) Normal—Walks 6 m (20 ft) in less than 5.5 seconds, no assistive devices, good speed, no evidence for imbalance, normal gait pattern, deviates no more than 15.24 cm (6 in) outside of the 30.48-cm (12-in) walkway width.
(2) Mild impairment—Walks 6 m (20 ft) in less than 7 seconds but greater than 5.5 seconds, uses assistive device, slower speed, mild gait deviations, or deviates 15.24-25.4 cm (6-10 in) outside of the 30.48-cm (12-in) walkway width.
(1) Moderate impairment—Walks 6 m (20 ft), slow speed, abnormal gait pattern, evidence for imbalance, or deviates 25.4-38.1 cm (10-15 in) outside of the 30.48-cm (12-in) walkway width. Requires more than 7 seconds to ambulate 6 m (20 ft).
(0) Severe impairment—Cannot walk 6 m (20 ft) without assistance, severe gait deviations or imbalance, deviates greater than 38.1 cm (15 in) outside of the 30.48-cm (12-in) walkway width or reaches and touches the wall.

### ______2. Change in Gait Speed

Instructions: Begin walking at your normal pace (for 1.5 m [5 ft]). When I tell you “go,” walk as fast as you can (for 1.5 m [5 ft]). When I tell you “slow,” walk as slowly as you can (for 1.5 m [5 ft]).
Grading: Mark the highest category that applies.
(3) Normal—Able to smoothly change walking speed without loss of balance or gait deviation. Shows a significant difference in walking speeds between normal, fast, and slow speeds. Deviates no more than 15.24 cm (6 in) outside of the 30.48-cm (12-in) walkway width.
(2) Mild impairments—Is able to change speed but demonstrates mild gait deviations, deviates 15.24-25.4 cm (6-10 in) outside of the 30.48-cm (12-in) walkway width, or no gait deviations but unable to achieve a significant change in velocity, or uses an assistive device.
(1) Moderate impairment—Makes only minor adjustments to walking speed, or accomplishes a change in speed with significant gait deviations, deviates 25.4-38.1 cm (10-15 in) outside the 30.48-cm (12-in) walkway width, or changes speed but loses balance but is able to recover and continue walking.
(0) Severe impairment—Cannot change speeds, deviates greater than 38.1 cm (15 in) outside 30.48-cm (12-in) walkway width, or loses balance and has to reach for wall or be caught.
   ______3. Gait with Horizontal Head Turns
Instructions: Walk from here to the next mark 6 m (20ft) away. Begin walking at your normal pace. Keep walking straight; after 3 steps, turn your head to the right and keep walking straight while looking to the right. After 3 more steps, turn your head to the left and keep walking straight while looking left. Continue alternating looking right and left every 3 steps until you have completed 2 repetitions in each direction.
Grading: Mark the highest category that applies.
(3) Normal—Performs head turns smoothly with no change in gait. Deviates no more than 15.24 cm (6 in) outside 30.48-cm (12-in) walkway width.
(2) Mild impairment—Performs head turns smoothly with slight change in gait velocity (eg, minor disruption to smooth gait path), deviates 15.24-25.4 cm (6-10 in) outside 30.48-cm (12-in) walkway width, or uses an assistive device.
(1) Moderate impairment—Performs head turns with moderate change in gait velocity, slows down, deviates 25.4-38.1 cm (10-15 in) outside 30.48-cm (12-in) walkway width but recovers, can continue to walk.
(0) Severe impairment—Performs task with severe disruption of gait (eg, staggers 38.1 cm [15 in] outside 30.48-cm (12-in) walkway width, loses balance, stops, or reaches for wall).
   ______4. Gait with Vertical Head Turns
Instructions: Walk from here to the next mark (6 m [20 ft]). Begin walking at your normal pace. Keep walking straight; after 3 steps, tip your head up and keep walking straight while looking up. After 3 more steps, tip your head down, keep walking straight while looking down. Continue alternating looking up and down every 3 steps until you have completed 2 repetitions in each direction.
Grading: Mark the highest category that applies.
(3) Normal—Performs head turns with no change in gait. Deviates no more than 15.24 cm (6 in) outside 30.48-cm (12-in) walkway width.
(2) Mild impairment—Performs task with slight change in gait velocity (eg, minor disruption to smooth gait path), deviates 15.24-25.4 cm (6-10 in) outside 30.48-cm (12-in) walkway width or uses assistive device.
(1) Moderate impairment—Performs task with moderate change in gait velocity, slows down, deviates 25.4-38.1 cm (10-15 in) outside 30.48-cm (12-in) walkway width but recovers, can continue to walk.
(0) Severe impairment—Performs task with severe disruption of gait (eg, staggers 38.1 cm [15 in] outside 30.48-cm (12-in) walkway width, loses balance, stops, reaches for wall).

### ______5. Gait and Pivot Turn

Instructions: Begin with walking at your normal pace. When I tell you, “turn and stop, ” turn as quickly as you can to face the opposite direction and stop.
Grading: Mark the highest category that applies.
(3) Normal—Pivot turns safely within 3 seconds and stops quickly with no loss of balance.
(2) Mild impairment—Pivot turns safely in >3 seconds and stops with no loss of balance, or pivot turns safely within 3 seconds and stops with mild imbalance, requires small steps to catch balance.
(1) Moderate impairment—Turns slowly, requires verbal cueing, or requires several small steps to catch balance following turn and stop.
(0) Severe impairment—Cannot turn safely, requires assistance to turn and stop.
   ______6. Step over Obstacle
Instructions: Begin walking at your normal speed. When you come to the shoe box, step over it, not around it, and keep walking.
Grading: Mark the highest category that applies.
(3) Normal—Is able to step over 2 stacked shoe boxes taped together (22.86 cm [9 in] total height) without changing gait speed; no evidence of imbalance.
(2) Mild impairment—Is able to step over one shoe box (11.43 cm [4.5 in] total height) without changing gait speed; no evidence of imbalance.
(1) Moderate impairment—Is able to step over one shoe box (11.43 cm [4.5 in] total height) but must slow down and adjust steps to clear box safely. May require verbal cueing.
(0) Severe impairment—Cannot perform without assistance.
   ______7. Gait with Narrow Base of Support
Instructions: Walk on the floor with arms folded across the chest, feet aligned heel to toe in tandem for a distance of 3.6 m [12 ft]. The number of steps taken in a straight line are counted for a maximum of 10 steps.
Grading: Mark the highest category that applies.
(3) Normal—Is able to ambulate for 10 steps heel to toe with no staggering.
(2) Mild impairment—Ambulates 7-9 steps.
(1) Moderate impairment—Ambulates 4-7 steps.
(0) Severe impairment—Ambulates less than 4 steps heel to toe or cannot perform without assistance.
   ______8. Gait with Eyes Closed
Instructions: Walk at your normal speed from here to the next mark (6 m [20 ft]) with your eyes closed.
Grading: Mark the highest category that applies.
(3) Normal—Walks 6 m (20 ft), no assistive devices, good speed, no evidence of imbalance, normal gait pattern, deviates no more than 15.24 cm (6 in) outside 30.48-cm (12-in) walkway width. Ambulates 6 m (20 ft) in less than 7 seconds.
(2) Mild impairment—Walks 6 m (20 ft), uses assistive device, slower speed, mild gait deviations, deviates 15.24-25.4 cm (6-10 in) outside 30.48-cm (12-in) walkway width. Ambulates 6 m (20 ft) in less than 9 seconds but greater than 7 seconds.
(1) Moderate impairment—Walks 6 m (20 ft), slow speed, abnormal gait pattern, evidence for imbalance, deviates 25.4-38.1 cm (10-15 in) outside 30.48-cm (12-in) walkway width. Requires more than 9 seconds to ambulate 6 m (20 ft).
(0) Severe impairment—Cannot walk 6 m (20 ft) without assistance, severe gait deviations or imbalance, deviates greater than 38.1 cm (15 in) outside 30.48-cm (12-in) walkway width or will not attempt task.

### ______9. Ambulating Backwards

Instructions: Walk backwards until I tell you to stop.
Grading: Mark the highest category that applies.
(3) Normal—Walks 6 m (20 ft), no assistive devices, good speed, no evidence for imbalance, normal gait pattern, deviates no more than 15.24 cm (6 in) outside 30.48-cm (12-in) walkway width.
(2) Mild impairment—Walks 6 m (20 ft), uses assistive device, slower speed, mild gait deviations, deviates 15.24-25.4 cm (6-10 in) outside 30.48-cm (12-in) walkway width.
(1) Moderate impairment—Walks 6 m (20 ft), slow speed, abnormal gait pattern, evidence for imbalance, deviates 25.4-38.1 cm (10-15 in) outside 30.48-cm (12-in) walkway width.
(0) Severe impairment—Cannot walk 6 m (20 ft) without assistance, severe gait deviations or imbalance, deviates greater than 38.1 cm (15 in) outside 30.48-cm (12-in) walkway width or will not attempt task.

### ______10. Steps

Instructions: Walk up these stairs as you would at home (ie, using the rail if necessary). At the top turn around and walk down.
Grading: Mark the highest category that applies.
(3) Normal—Alternating feet, no rail.
(2) Mild impairment—Alternating feet, must use rail.
(1) Moderate impairment—Two feet to a stair; must use rail.
(0) Severe impairment—Cannot do safely.

### Clinical Significance

The Functional Gait Assessment (FGA) is an evaluation tool used by therapists designed to measure a patient's ability to perform dynamic balance tasks while walking. It is designed specifically for patients with vestibular disorders. It rates performance from 0 (poor) to 3 (excellent) on ten different gait tasks including gait on a level surface, gait when changing speeds, gait and head turns in a vertical or horizontal direction, gait with pivot turn, stepping over an obstacle, gait with a narrow base of support, gait with eyes closed, ambulating backwards and steps. Scores on the Functional Gait Assessment range from 0 to 30. The FGA is based on the Dynamic Gait Index (DGI), and was developed to avoid the ceiling effect sometimes seen with patients with vestibular disorders when evaluated by the DGI and to provide more clear scoring instructions for the evaluating therapist.

Reliability, Internal Consistency, and Validity of Data Obtained with the Functional Gait Assessment. Diane M Wrisley, Gregory F Marchetti, Diane K Kuharsky, Susan L Whitney. Physical Therapy; October 2004; 84, 10; Health Module p. 906-918.

### Berg Balance Scale

Description: The Berg Balance Scale is an objective measure of static and dynamic balance activities. The test has been used to identify and evaluate balance impairment in the elderly.
Equipment needed: Chair with arms rests
   Chair without arm rests
   Stop watch
   Ruler (0-10 inches)
   A shoe
   6-inch tall foot stool
Completion Time: 20 minutes
Scoring: A five-point ordinal scale, ranging from 0-4. “0” indicates the lowest level of function and “4” the highest level of function.
Interpretation: Score ranges from 0-56, a score of <46 indicates a high risk of falls.
Berg Balance Scale Do not use an assistive device with any component of this test
1. ______ Sitting to Standing Instructions: Please stand up. Try not to use your hands for support.
   4 Able to stand without using hands and stabilize independently
   3 Able to stand independently using hands
   2 Able to stand using hands after >1 tries
   1 Needs minimal assist to stand or stabilize
   0 Needs moderate or maximal assist to stand
2. ______ Standing Unsupported Instructions: Please stand for two minutes without support.
   4 Able to stand safely 2 minutes
   3 Able to stand 2 minutes with supervision
   2 Able to stand 30 seconds unsupported
   1 Needs several tries to stand 30 seconds unsupported
   0 Unable to stand 30 seconds unassisted
If subject is able to stand 2 minutes unsupported, score full points for sitting unsupported. Proceed to item #4
3. ______ Sitting with back unsupported but feet on floor Instructions: Please sit with arms folded for 2 minutes.
   4 Able to sit safely and securely for 2 minutes
   3 Able to sit 2 minutes with supervision
   2 Able to sit 30 seconds
   1 Able to sit 10 seconds
   0 Unable to sit without support 10 seconds
4. ______ Standing to Sitting Instructions: Please sit down (do not cue to use UE's)
   4 Sits safely with minimal use of hands
   3 Controls descent by using hands
   2 Uses back of legs against chair to control descent
   1 Sits independently but has uncontrolled descent
   0 Needs assistance to sit
5. ______ Transfers Instructions: Arrange chair(s) for pivot transfer. Ask subject to transfer one way toward a seat with armrests and one way toward a seat without armrests. You may use tow chairs (one with and one without armrests) or a bed and a chair.
   4 Able to transfer safely with minor use of hands
   3 Able to transfer safely with definite use of hands
   2 Able to transfer with verbal cueing and/or supervision
   1 Needs one person to assist
   0 Needs two people to assist or supervise to be safe
6. ______ Standing unsupported with eyes closed Instructions: Please close your eyes and stand still for 10 seconds.
   4 Able to stand 10 seconds safely
   3 Able to stand 10 seconds with supervision
   2 Able to stand 3 seconds
   1 Unable to keep eyes closed 3 seconds but stays steady
   0 Needs help to keep from falling
7. ______ Standing unsupported with feet together Instructions: Place your feet close together and stand without holding.
   4 Able to place feet together independently and stand 1 minute safely
   3 Able to place feet together independently and stand 1 minute with supervision
   2 Able to place feet together independently but unable to hold for 30 seconds
   1 Needs help to attain position but able to stand 15 seconds feet together
   0 Needs help to attain position and unable to stand for 15 seconds
8. ______ Reaching forward with outstretched arm while standing Instructions: Lift arm to shoulder level. Stretch out your fingers and reach forward as far as you can. (Examiner places a ruler at end of fingertips when arm is at 90 degrees. Fingers should not touch the ruler while reaching forward. The recorded measure is the distance forward that the fingers reach while the subject is in the most forward lean position. When possible, ask the subject to use both arms when reaching to avoid rotation of the trunk)
   4 Can reach forward confidently >25 cm (10 inches)
   3 Can reach forward >12 cm (5 inches)
   2 Can reach forward >5 cm (2 inches)
   1 Reaches forward but requires supervision
   0 Loses balance while trying/requires external support
90. ______ From a standing position pick up object from the floor Instructions: Pick up an object (the size of a shoe), which is placed in front of your feet.
   4 Able to pick up object safely and easily
   3 Able to pick up object but needs supervision
   2 Unable to pick up but reaches 2-5 cm (1-2 inches) from shoe and keeps balance independently
   1 Unable to pick up and needs supervision while trying
   0 Unable to try/needs assist to keep from losing balance or falling
10. ______ Turning to look behind over left & right shoulders while standing Instructions: Turn to look directly behind you over your left shoulder. Repeat to the right. Examiner may pick an object to look at directly behind the subject to encourage a better turn.
   4 Looks behind from both sides and shifts weight well
   3 Looks behind one side only, other side shows less weight shift
   2 turns sideways only, but maintains balance
   1 Needs supervision when turning
   0 Needs assist to keep from losing balance or falling
1 . ______ Turn 360 degrees Instructions: turn completely around in a full circle. Pause. Then turn a full circle in the other direction.
   4 Able to turn 360 degrees safely in 4 seconds or less
   3 Able to turn 360 degrees safely one side only in 4 seconds or less
   2 Able to turn 360 degrees safely but slowly (greater than 4 seconds)
   1 Needs close supervision or verbal cueing
   0 needs assistance while turning
12. ______ Placing alternate foot on step or stool while standing unsupported Instructions: Place each foot alternately on the step/stool. Continue until each foot has touched the step/stool 4 times. Use a 6-inch step stool against the wall. Foot flat on stool and back down, not just toe touch)
   4 Able to stand independently and safely complete 8 steps in 20 seconds
   3 Able to stand independently and complete 8 steps in >20 seconds
   2 Able to complete 4 steps without aid, with supervision
   1 Able to complete >2 steps, needs minimal assist
   0 Needs assistance to keep from falling/unable to try
13. ______ Standing unsupported one foot in front Instructions: Place one foot directly in front of the other. If you feel you can not place your foot directly in front of you, try to step far enough ahead that the heel of your forward foot is ahead of the toes of the other foot. (To score 3 points, the length of the step should exceed the length of the other foot and the width of the stance should approximate the subject normal stride width.)
   4 Able to place foot strict tandem independently and hold 30 seconds
   3 Able to place foot ahead of other (semi-tandem) independently and hold 30 seconds
   2 Able to take small step independently and hold 30 seconds
   1 Needs help to step but can hold 15 seconds
   0 Loses balance while stepping or standing
14. ______ Standing on one leg Instructions: Stand on one leg as long as you can without holding. (subject can choose which leg)
   4 Able to lift leg independently and hold >10 seconds
   3 Able to lift leg independently and hold 5-10 seconds
   2 Able to lift leg independently and hold =or <3 seconds
   1 Tried to lift leg, unable to hold 3 seconds but remains standing independently
   0 Unable to try or needs assist to prevent fall

### Clinical Significance

The Berg Balance Scale is an evaluation tool used by therapists designed to measure functional balance. The items test the subject's ability to maintain balance in positions or movements of increasing difficulty and the ability of the subject to change positions. The scale rates performance from 0 (poor) to 4 (excellent) on fourteen common tasks including moving sit to/from stand, standing or sitting unsupported, transfers, standing with eyes closed, standing with a feet together, standing and reaching, retrieving an object from the ground, turning, alternate step-ups, tandem stand and single leg stance. Scores on the Berg Balance Scale range from 0 to 56, a score of <46 indicates an increased risk of falls.

Berg K O, Wood-Dauphinee S L, Williams J I, Maki B E. Measuring Balance in the Elderly: Preliminary Development of an Instrument. Physiotherapy Canada. 1989; 41(6): 304-311.

Berg K, Wood-Dauphinee S L, Williams J I, Maki B E. Measuring balance in the elderly: validation of an instrument. Can J Public Health 1992; 83: S7-S10.

Berg K O, Maki B E, Williams J I, Holliday P J, Wood-Dauphinee S L. Clinical and laboratory measures of postural balance in an elderly population. Arch Phys Med Rehabil 1992; 73: 1073-1080.

Berg K, Wood-Dauphinee S, Williams J I. The Balance Scale: reliability assessment with elderly residents and patient with an acute stroke. Scand J Rehabil Med 1994; 27: 27-36.

Harada N, Chiu V, Damron-Rodriguez J, et al. Screening for balance and mobility impairment in elderly individuals living in residential care facilities. PT 1995; 75: 462-469.

Russo S G. Clinical balance measures: literature resources. Neurology Report. 1997: 21 (1) :29-36.

### Timed Up and Go

Description: The Timed Up and Go is a functional measure of basic balance and mobility in the elderly.
Equipment needed: Armchair
   Stopwatch
   3 meter walkway (9.84 feet)
Completion Time: Up to 5 minutes
Scoring: The time taken in seconds required to complete the task.
Interpretation: <1 1.2 sec is correlated with falls in people with vestibular dysfunction. (Whitney et el)
Test instructions: “When I say go, you will stand from the chair, walk to the mark/cone on the floor, turn around, walk back to the chair and sit down. I will be timing you using the stopwatch.”
   Subject starts with their back against the back of the chair, their arms on the arm rests.
   Subjects should use assistive devices during the test if the typically use them. Indicate use of assistive device with the score.
   No physical assistance is given during the test.

Podsiadlo D, Richardson S. The timed “Up & Go”: A test of basic functional mobility for frail elderly persons. Journal of the American Geriatrics Society. 1991. 9: 142-148.

Russo S G. Clinical balance measures: literature resources. Neurology Report. 1997. 21 (1): 29-36.

Steffen T M, Hacker T A, Mollinger L. Age- and Gender-Related Test Performance in Community-Dwelling Elderly People: Six-Minute Walk Test, Berg Balance Scale, Timed Up & Go Test, and Gait Speeds. Physical Therapy. 2002. Vol 82(2): 128-137.

Whitney S L, Marchetti G F, Schade A, Wrisley D M. Journal of Vestibular Research. 2004. 14: 397-409.

### The Motion Sensitivity Test

Description: The Motion Sensitivity Test measures motion provoked dizziness during a series of quick changes to head or body position.
Equipment needed: Stopwatch
   Calculator
   Chair
   Bed or table that subject can lie down on
Completion Time: 15-20 minutes
Scoring: In each position the duration of the dizziness is timed in seconds and the subject rates the intensity on a 0-5 scale. The information is computed in a formula (see below) to receive a final score.
Interpretation: Score varies from 0-100. 0 indicates no symptoms, 100 indicates severe unrelenting symptoms in all positions.

| Baseline Symptoms | Intensity | Duration | Score |
|---|---|---|---|
| Resting Symptoms | | n/a | n/a |
| 1. Sitting to supine | | | |
| 2. Supine to left side | | | |
| 3. Supine to right side | | | |
| 4. Supine to sitting | | | |
| 5. Left hallpike | | | |
| 6. Up from left | | | |
| 7. Right hallpike | | | |
| 8. Up from right | | | |
| 9. Sitting, head tipped to left | | | |
| knee | | | |
| 10. Head up from left knee | | | |
| 11. Sitting, head tipped to | | | |
| right knee | | | |
| 12. Head up from right knee | | | |
| 13. Sitting, head turns 5 | | | |
| times, 180 degrees | | | |
| 14. Sitting, head tilts, 5 | | | |
| times | | | |
| 15. 180 degree turn to right, | | | |
| standing | | | |
| 16. 180 degree turn to left, | | | |
| standing | | | |

Scoring:
   1. Duration of dizziness (timed with a stopwatch) is assigned the following values:

| | |
|---|---|
| 1 point | 5-10 sec of dizziness |
| 2 points | 11-30 seconds of dizziness |
| 3 points | >30 seconds of dizziness |

2. After duration is determined, the subject rates the intensity:
   0-5 scale (0=no symptoms, 5=severe dizziness)
   Note: The subject rates dizziness intensity at rest, the intensity score at rest is subtracted from the intensity score at each position.
3. Duration and intensity points are added together.
4. A final score is calculated with the following formula:

[(Σ(duration+intensity)×number of dizziness provoking positions)/2048]×100

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method for objectively scoring and/or assessing balance function in a subject comprising:
a) providing a subject;
b) instructing the subject to perform one or more physical tests;
c) monitoring motion and/or movement of one or more locations present on said subject during performance of the physical tests, wherein monitoring motion and/or movement comprises use of a balance function data collection and analysis system; and
d) correlating the subject's motion and/or movement to one or more balance function footprints to provide the subject a score and/or assessment of the subject's balance function or a diagnosis of a subject based on their balance function or balance function data, compared to a balance function footprint.

2. The method of claim 1, wherein said balance function data collection and analysis system is configured to detect, transmit, store, process and assess balance function data.

3. The method of claim 2, wherein detecting and transmitting balance function data comprises use of one or more motion sensors.

4. The method of claim 2, wherein storing balance function data comprises placement of balance function data on storage means.

5. The method of claim 2, wherein processing and assessing balance function data comprises use of a computer processor running software configured to analyze and assess the balance function data.

6. The method of claim 1, wherein said subject is instructed to perform one or more tasks present within one or more tests, wherein said one or more tests are selected from the group consisting of dynamic gait index test, functional gait assessment test, Berg balance test, timed up and go test, and the dynamic task test.

7. A method for generating a balance function footprint comprising:
a) providing a plurality of subjects, wherein each subject of the plurality of subjects is identified as having a certain, common etiology;
b) a balance function data collection and analysis system, wherein said system is configured to detect, transmit, store, process and assess balance function data, wherein detecting and transmitting balance function data comprises use of one or more motion sensors; wherein storing balance function data comprises placement of balance function data on storage means; and processing and assessing balance function data comprises use of a computer processor running software configured to analyze and assess the balance function data;
c) instructing each subject of the plurality of subjects to perform an identical series of tasks,
d) using the balance function data collection and analysis system to
i) acquire motion and/or movement data from the subjects during the subject's performance of the task; and
ii) standardize the balance function data acquired to generate a balance function footprint.

8. A system comprising:
a) a database comprising one or more balance function footprints;
b) one or more sensors.

9. The system of claim 8, wherein said sensors are configured to transfer and/or transduce balance function data to a data storage device.

10. The system of claim 9, further comprising a processor and software for analyzing and/or characterizing said balance function data.

11. The system of claim 10, wherein said analyzing and/or characterizing provides a score and/or assessment to a subject regarding said subject's balance function.

12. The system of claim 8, wherein said sensors comprise sensors configured to detect motion of a subject at one or more points of said subject.

13. The system of claim 9, wherein balance function data is acquired from a subject tasked with performing one or more physical acts.
